(19) United States
(12) Patent Application Publication
(10) Pub. No.: US 2009/0170131 A1
(43) Pub. Date: Jul. 2, 2009

(54) Agents for Imaging Apoptosis

(76) Inventors: Juri Gelovani, Missouri City, TX (US); William Tong, Houston, TX (US); Amer Najjar, Katy, TX (US); Chun Li, Missouri City, TX (US)

Correspondence Address: BAKER BOTTS, LLP, 910 LOUISIANA, HOUSTON, TX, 77002-4995, US

(21) Appl. No.: 12/298,386

(22) PCT Filed: May 9, 2007
(86) PCT No.: PCT/US07/68596

§ 371 Date: Oct. 24, 2008

Related U.S. Application Data
(60) Provisional application No. 60/746,834, filed on May 9, 2006.

(51) Int. Cl. G01N 33/53

(52) U.S. Cl. 435/7.23; 435/7.21

(57) ABSTRACT

Compositions, and methods of using such compositions, having the following Formula (I) or salt, ester, or hydrate thereof, wherein R₁ is Asp-Glu peotide, tert-butyloxycarbonyl, methoxyphenylacetyl, bromomethoxyphenylaceityl, fluoromethoxyphenylacetyl, (methylmethoxyphenyl)acetyl, or iodomethoxyphenylacetyl; R₂ is valine with or without protecting groups), aspartale (with or without protecting groups), 1-Butyl, hydrogen, aniline, aromatic molecules, tert-butyloxycarbonyl, or fluorenyl-methoxy-carbonyl; and R₃ is p-nitroaniline, 7-amino-4-methylcoumarin, fluoroethylamine, 1-amino-4-fluoromethyl-cyclohexane, 1-amino-4-fluoroethyl-cyclohexane, aniline, p-fluoroaniline, p-fluoromethylaniline, p-fluorobenzylamine, p-fluoromethylbenzylamine, 4-aminopiperidine, 4-amino-1-fluoroethylpiperidine, or 4-amino-1-fluoropropylpiperidine.

### CROSS-REFERENCE TO RELATED APPLICATIONS

This application claims the benefit of U.S. Provisional Application No. 60/746,834, filed May 9, 2006, which is incorporated herein by reference.

### BACKGROUND

Apoptosis, or programmed cell death, is a common property of all multicellular organisms. Apoptosis regulates cell number, facilitates morphogenesis, removes harmful or otherwise abnormal cells, and eliminates cells that have already performed their function. Additionally, apoptosis occurs in response to various physiological stresses, such as hypoxia or ischemia. It can be triggered by a number of factors, including ultraviolet or γ irradiation, growth factor withdrawal, chemotherapeutic drugs, or signaling by death receptors. Because many important diseases (e.g., cancer, AIDS, auto-immune diseases, and neurodegenerative diseases) are related to defective or excessive programmed cell death, drugs that can either facilitate or block programmed cell death are potentially useful in treating many diseases.

The central role in the regulation and the execution of apoptotic cell death belongs to caspases. Caspases, a family of cysteinyl aspartate-specific proteases, are synthesized as zymogens with a prodomain of variable length followed by a large subunit (p20) and a small subunit (p10). The caspases are activated through proteolysis at specific asparagine residues that are located within the prodomain, the p20 and p10 subunits. This results in the generation of mature active caspases that consist of the heterotetramer p202-p102. Subsequently, active caspases specifically process various substrates that are implicated in apoptosis and inflammation. The important role of caspases in these processes makes them attractive targets for drug development. Caspases also represent important targets for noninvasive molecular imaging to assess therapeutic efficacy after initiation of treatment.

Caspases are specific cysteine proteases, recognizing 4 amino acids, named P4-P3-P2-P1. The cleavage takes place typically after the C-terminal residue (P1), which is usually an aspartate. The preferred P3 position is an invariant glutamate for all mammalian caspases. Thus, specificity of caspase cleavage can be described as X-E-X-D (i.e., X-Glu-X-Asp). Caspase-1, -4, -5, -13, and -14 prefer the tetrapeptide sequence WEHD. Caspase-2, -3, and -7 have a preference for the substrate DEXD, whereas caspase-6, -8, and -9 prefer the sequence (L/V)EXD. The cleavage site between the large and small subunits for initiator caspases carries its own tetrapeptide recognition motif, which is consistent with the proposed mechanism of autoactivation of initiator caspases.

Most of the synthetic peptide caspase inhibitors were developed based on the tetrapeptide caspase recognition motif. The introduction of an aldehyde group at the C-terminus of the tetrapeptide results in the generation of reversible inhibitors, whereas a fluoromethyl ketone (fmk), a chloromethyl ketone (cmk), or a diazomethyl ketone (dmk) at this position irreversibly inactivates the enzyme.

At present, there exists a variety of techniques that can detect the process of apoptosis at different stages. For example, the terminal stage of apoptosis can be assayed by morphological changes of the cell (such as the presence of apoptotic bodies). Before that, apoptosis can be assayed by DNA fragmentation using either gel analysis or the TUNEL technique. Early stages of apoptosis can be assayed by the turnover of PS (phosphatidylserine) in the membrane using an Annexin V-FITC labeled protein, or by detecting the activation of caspase-3 using a fluorescent dye linking to a substrate peptide. All of these techniques, however, have certain limitations. For example, gel analysis can only be applied to an extract of cells, not to a single cell or intact cells. The TUNEL method can only be applied to fixed cells, not living cells. Annexin V can only detect events at the outer cell surface, not inside the cell. The caspasc probe using a peptidc linked fluorescence dye also has limitations. First, this probe cannot penetrate the cell membrane, and thus, it is typically used to assay cell extract. Secondly, the fluorescent change resulting from caspase cleavage involves mainly a shift of the emission spectrum in the dye rather than a total destruction of the fluorescence, and sensitivity is limited.

Molecular imaging agents for visualization and quantitation of caspase expression and activity by PET have demonstrated limited feasibility and clinical applicability. Previous attempts to image caspases with irreversible inhibitors like [¹³¹I]IZ-VAD-fmk failed. Although some increase in uptake of [¹³¹I]IZ-VAD-fmk was observed in cell cultures containing 23% apoptotic cells, these levels were not sufficient for in vivo imaging. The use of a caspase inhibitor may result in the trapping of one tracer molecule per activated caspase. Thus, the number of activated caspases may be too low to induce an accumulation of the radiolabeled caspase inhibitor at levels high enough to be used for imaging purposes.

### SUMMARY

The present disclosure, according to certain embodiments, is generally directed to compositions and methods for intracellular detection of enzymes. More particularly, the present disclosure is directed to agents for molecular imaging of caspases important in apoptosis and associated methods of use.

The present disclosure provides methods and compositions that overcome the deficiencies of current molecular imaging agents for visualization of caspase expression. The imaging agents of the present disclosure are capable of serving as a substrate based imaging agent for molecular imaging of caspases.

The use of a caspase inhibitor results in the trapping of one tracer molecule per activated caspase. Thus, the number of activated caspases is probably too low to induce an accumulation of the radiolabeled caspase inhibitor which is high enough to be used for imaging purposes. However, the application of substrate based imaging agent of the present disclosure for molecular imaging of caspases should be more effective and, at radiotracer concentrations, non-saturable. Because activated caspases are able to cleave multiple substrates, this should result in an amplification of the intracellular radioactivity and, therefore, in an enhancement of the imaging signal.

### DRAWINGS

Some specific example embodiments of the disclosure may be understood by referring, in part, to the following description and the accompanying drawings.

FIG. 1 is an image of AMC fluorescence from an imaging agent in DLD1 colon carcinoma cells before (A) and after 2 hour treatment with TNF-related apoptosis-inducing ligand (TRAIL) (B). The corresponding bright-field images demonstrate the correlation between fluorescence and the apoptotic state of the cells before (C) and after (D) TRAIL treatment.

FIG. 2 shows fluorescence intensity of an imaging agent measured from a representative region of interest within each 10 minute frame. The increase in fluorescence due to AMC-M808 cleavage during the first hour following TRAIL treatment was determined to proceed at rate of 5.5 min⁻¹. In the presence of DEVD-CHO, no net increase in fluorescence was observed due to caspase-3 activity inhibition.

FIG. 3 shows the alignment of two molecules, acetyl-DEVD-fluoroethylamide to dimethoxyphenylacetate-VD-fluoroethylamide.

FIG. 4 is a schematic diagram showing the products of cleavage of the imaging agent by active caspase-3 enzyme in a mammalian cell.

FIG. 5 is a schematic illustrating an example of radiosynthesis of imaging agent using synthon and stannylation chemistry.

FIG. 6 illustrates synthesis reactions resulting in the formation of an imaging agent using solid phase synthesis.

FIG. 7 illustrates synthesis reactions for radiofluorination and ¹¹C radiolabeling of an imaging agent.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure, according to certain embodiments, is generally directed to compositions and methods for intracellular detection of enzymes. More particularly, the present disclosure is directed to agents for molecular imaging of caspases important in apoptosis and associated methods of use.

The imaging agents of the present disclosure are capable of serving as a substrate based imaging agent for molecular imaging of caspases. The imaging agents of the present disclosure should be lipophilic enough to cross cell membranes by non-facilitated diffusion so that they can freely cross in and, most importantly, out of a cell (in contrast to unidirectional Tat or Perm1-mediated internalization). Furthermore, the imaging agents should be recognized by a specific caspase (e.g., caspase-3) and be cleaved at the Asp amine to release a labeled product, which should ideally be retained and accumulated within the cell by being unable to cross the cell membrane, binding to intracellular macromolecules, or at least effluxing very slowly from the cell. In addition, the rate of transmembrane passage of this compound should not be a rate-limiting factor in the process of retention of the cleaved labeled group. Finally, imaging agents generally should be at least partially resistant to systemic metabolic degradation. As used herein, the term “labeled” refers to any component or portion of a compound that is capable of producing a detectable signal.

The imaging agents generally serve as a substrate to caspases by utilizing cleavage site peptide sequences from known or potential natural substrates of caspase enzymes. Table 1 lists some peptide sequences corresponding to known or potential cleavage sites that may be natural substrates for caspases. The imaging agents may be designed as a caspase substrate using the peptide sequences listed in Table 1. Thus, imaging agents of the present disclosure may be designed to have specificity for any caspase. The imaging agent may also be designed to measure more than one enzyme at. a time, by designing substrates that are recognized and cleaved by more than one of the enzymes involved in the caspase cascade.

TABLE 1

| | Caspase | Substrate Specificity |
|---|---|---|
| Group I | Caspase-1 | WEHD |
| | Caspase-4 | (W/L)EHD |
| | Caspase-5 | (W/L)EHD |
| | Caspase-13 | WEHD |
| | Caspase-14 | WEHD |
| Group II | Caspase-2 | DEHD |
| | Caspase-8 | LETD |
| | Caspase-9 | LEHD |
| | Caspase-10 | LEXD |
| Group III | Caspase-3 | DEVD |
| | Caspase-6 | VEHD |
| | Caspase-7 | DEVD |

In certain embodiments, the above-described imaging agents may comprise labeled compounds represented by the following structure: R₁-V-D-(O-R₂)-R₃ ( i.e., R₁-Val-Asp-(O-R₂)-R₃). Such imaging agents are lipophilic enough to enter a cell by passive diffusion through the cell membrane. Once inside the cell, cleavage of the imaging agent occurs at the Asp amine by an active caspase enzyme. This cleavage results in the formation of at least one labeled product. In some cases, the cleavage may result in an unlabeled product. The labeled product will generally remain in the cell, while any unlabeled product is free to diffuse out of the cell or remain in the cell, depending on its lipophilicity.

Lipophilicity may be determined with reference to the octanol-water distribution coefficient (logD) at physiological pH, typically 7.4. The octanol-water distribution coefficient is the log ratio of the sum of the concentrations of all species of the compound in octanol to the sum of the concentrations of all species of the compound in water. It is a measure of the lipophilicity of a compound. Suitable imaging agents generally have a logD greater than zero to facilitate permeability and diffusion of the molecule through the cell membrane.

To detect the presence of the labeled product within a cell, the imaging agent may be radiolabeled such that when it is cleaved by a caspase, the labeled product is similarly radiolabeled. Accumulation of such a labeled product in a cell may be imaged indicating the level of caspase activity. Radiolabeling of the intact imaging agent generally occurs at either the R₁ or R₃ position. Generally, the position of the radiolabeled group depends on the logD of the labeled product. The position of the radiolabeled group is chosen to result in a labeled product with a logD sufficient to allow accumulation of the labeled product within a cell. In some examples, the labeled product may have a logD that is greater than zero. And unlabeled product, if any, may have a logD greater than zero or less than zero. Examples of suitable groups that may be used to radiolabel the imaging agent are radioisotopes of carbon (e.g., ¹¹C), bromine (e.g., ⁷⁶Br), fluorine (e.g., ¹⁸F), and iodine (e.g., ¹²³I, ¹²⁴I, ¹³¹I).

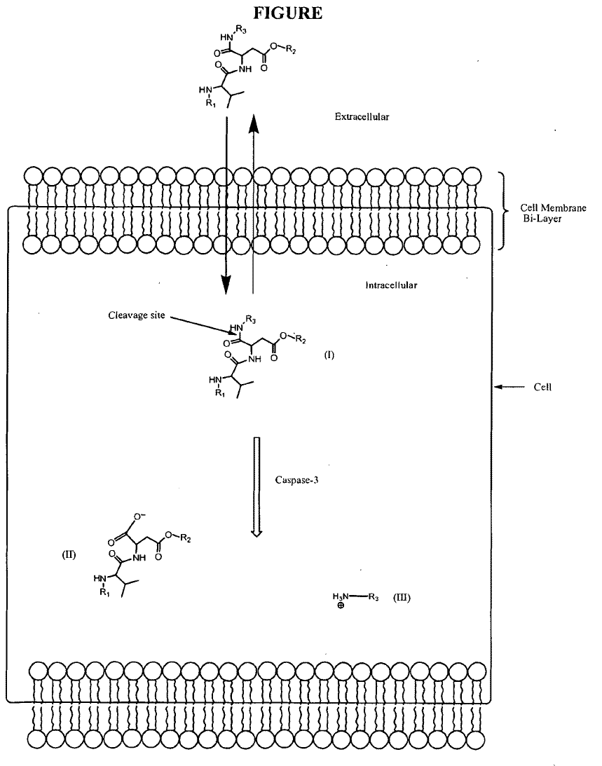

FIG. 4 illustrates one example of the process of cleavage of an imaging agent by active caspase-3 enzyme and the formation of the resulting cleavage products. For example, referring to FIG. 4, the imaging agent (1) enters the cells by diffusion. The logD of the imaging agent should be greater than zero to facilitate permeability and diffusion of the imaging agent through the cell. Once inside the cell, active caspase-3 recognizes the imaging agent as its substrate and cleaves the agent at the Asp amine group. The cleavage results in the formation of two cleavage products (II) and (III). In certain examples, R₁ may contain the radiolabeled group. In these examples, the logD of cleavage product (II) should be less than zero, thereby allowing for cleavage product (II) to accumulate within the cells. In other examples, R₃ may contain the radiolabeled group, and the logD of cleavage product (III) may be less than zero to allow for the accumulation of cleavage product (III) within the cells.

In another example, still referring to FIG. 4, cleavage product (III) may contain a radiolabeled group at the R₃ position, but may have a logD greater than zero. In these instances, the lysosomes of the cell aid in the accumulation of cleavage product (III) within in the cell. Lysosomes are organelles of the cell that contain enzymes required for intracellular digestion. Compounds containing amine groups, such as cleavage product (III), can be trapped within the lysosome, thereby allowing for accumulation within the cell.

To detect the presence of the labeled product within a cell, the imaging agent may bc labeled with a fluorescent group. The fluoresccnt group may bc present on the imaging agent at the R₁ or the R₃ position. The intact imaging agent quenches the fluorescence of the fluorescent group in the R₁ or the R₃ position. When allowed to enter a cell, however, active caspase cleaves the imaging agent at the terminal Asp amine group, thereby allowing the fluorescent group to respond with a large increase in fluorescence emission, producing a detectable signal (e.g., via fluorescent resonance energy transfer, FRET). The group capable of fluorescence may be a portion of the labeled cleavage product having a logD less than zero, and thus accumulates in the cells or tissues, thereby allowing enhanced detection of caspase activity. In certain examples, the group capable of fluorescence may be in the R₃ position of the labeled product and may have a logD greater than zero. These cleavage products, despite their increased logD, may still be retained in the cells due to trapping of the product by the lysosomcs.

For example, referring to FIG. 4, the imaging agent containing a group capable of fluorescence (I) enters the cells by diffusion. The logD of the imaging agent should be greater than zero to facilitate permeability and diffusion of the imaging agent through the cell. Once inside the cell, active caspase-3 recognizes the imaging agent as its substrate and cleaves the agent at the Asp amine group. The cleavage results in the formation of two cleavage products (II) and (III). In certain examples, R₁ may contain the fluorescent group, and the logD of cleavage product (II) should be less than zero, thereby allowing for cleavage product (II) to accumulate within the cells. In other examples, R₃ contains the fluorescent group, and the logD of cleavage product (III) may be less than zero to allow for the accumulation of cleavage product (III) within the cells. In certain examples, R₃ contains the fluorescent group and cleavage product (III) has a logD that is greater than zero. The cleavage product (III) may still remain in the cells as a result of the trapping of the product within the lysosomes of the cells.

In certain embodiments, imaging agents of the present disclosure comprise the following formula (I):

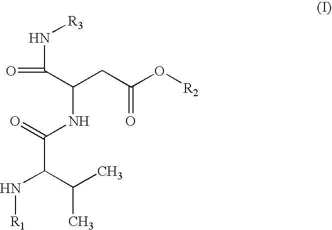

wherein R₁ represents an Asp-Glu peptide, tert-butyloxycarbonyl (BOC), methoxyphenylacetyl, bromomethoxyphenylacetyl, fluoromethoxyphenylacetyl, (methylmethoxyphenyl)acetyl and iodomethoxyphenylacetyl, and may optionally be radiolabeled; R₂ represents any group that can modulate the lipophilicity of the imaging agent to allow for passive diffusion of the imaging agent into the cell, such as, for example, t-Butyl, hydrogen, aniline, aromatic molecules, BOC, fluorenyl-methoxy-carbonyl (Fmoc), valine (with or without protecting groups), and aspartate (with or without protecting groups); R₃ represents p-nitroaniline, 7-amino4-methylcoumarin (AMC), fluoroethylamine, 1-amino-4-fluoromethyl-cyclohexane, 1-amino-4-fluoroethyl-cyclohexane, aniline, p-fluoroaniline, p-fluoromethylanil ne, p-fluorobenzylamine, p-fluoromethylbenzylamine, 4-aminopiperidine, 4-amino-l-fluoroethylpiperidine, and 4-amino-1-fluoropropylpiperidine, and optionally may be radiolabeled.

The compound may be a salt, ester, or hydrate thereof.

Examples of imaging agents according to Formula I include, but are not limited, the compounds listed in Table 2.

The imaging agents represented by Formula I are cell-permeable, that is, they can be introduced into whole cells or tissue samples and are lipophilic enough to passively diffuse through the cell membrane. In determining suitable R groups, logD may be assessed for the imaging agent. A suitable imaging agent would have a logD greater than zero. Table 2 shows the calculated logD for the cleaved products of example imaging agents and the logD for example intact imaging agents.

TABLE 2

| | | | | Substrate | Cleaved LogD | | Radiolabeling | |
|---|---|---|---|---|---|---|---|---|
| No. | R₁ | R₂ | R₃ | LogD | R₁-R₂ | R₃ | R₁-R₂ | R₃ |
| 1 | BOC | t-Butyl | p-nitroaniline | 5.26 | | 1.39 | | |
| 2 | BOC | H | p-nitroaniline | 0.33 | | 1.39 | | |
| 3 | (methoxyphenyl)acetyl | Benzyl | AMC | 5.83 | −3.16 | 1.13 | | |
| 4 | (methoxyphenyl)acetyl | H | AMC | 0.36 | −3.16 | 1.13 | | |
| 5 | (methoxyphenyl)acetyl | H | fluoroethylamine | −1.79 | −3.16 | −0.72 | ¹¹C | ¹⁸F |
| 6 | (bromomethoxyphenyl)acetyl | H | fluoroethylamine | −0.⁷⁶ | −2.13 | −0.72 | ⁷⁶Br, | ¹⁸F |
| | | | | | | | ¹¹C | |
| 7 | (iodomethoxyphenyl)acetyl | H | fluoroethylamine | −0.53 | −1.91 | −0.72 | ¹²³I, ¹²⁴I, ¹³¹I, | ¹⁸F |
| | | | | | | | ¹¹C | |
| 8 | (methoxyphenyl)acetyl | t-Butyl | fluoroethylamine | 3.17 | −0.48 | −0.72 | ¹¹C | ¹⁸F |
| 9 | (bromomethoxyphenyl)acetyl | t-Butyl | fluoroethylamine | 4.2 | 0.55 | −0.72 | ⁷⁶Br, | ¹⁸F |
| | | | | | | | ¹¹C | |
| 10 | (iodomethoxyphenyl)acetyl | t-Butyl | fluoroethylamine | 4.42 | 0.77 | −0.72 | ¹²³I, ¹²⁴I, ¹³¹I, | ¹⁸F |
| | | | | | | | ¹¹C | |
| 11 | (methoxyphenyl)acetyl | H | 1-amino-4-fluoromethyl- | −0.33 | −3.16 | −1.66 | ¹¹C | ¹⁸F |
| | | | cyclohexane | | | | | |
| 12 | (bromomethoxyphenyl)acetyl | H | 1-amino-4-fluoromethyl- | 0.7 | −2.13 | −1.66 | ⁷⁶Br, | ¹⁸F |
| | | | cyclohexane | | | | ¹¹C | |
| 13 | (iodomethoxyphenyl)acetyl | H | 1-amino-4-fluoromethyl- | 0.93 | −1.91 | −1.66 | ¹²³I, ¹²⁴I, ¹³¹I, | ¹⁸F |
| | | | cyclohexane | | | | ¹¹C | |
| 14 | (methoxyphenyl)acetyl | t-Butyl | 1-amino-4-fluoromethyl- | 4.6 | −0.48 | −1.66 | ¹¹C | ¹⁸F |
| | | | cyclohexane | | | | | |
| 15 | (bromomethoxyphenyl)acetyl | t-Butyl | 1-amino-4-fluoromethyl- | 5.63 | 0.55 | −1.66 | ⁷⁶Br, | ¹⁸F |
| | | | cyclohexane | | | | ¹¹C | |
| 16 | (iodomethoxyphenyl)acetyl | t-Butyl | 1-amino-4-fluoromethyl- | 5.86 | 0.77 | −1.66 | ¹²³I, ¹²⁴I, ¹³¹I, | ¹⁸F |
| | | | cyclohexane | | | | ¹¹C | |
| 17 | (methoxyphenyl)acetyl | H | 1-amino4-fluoroethyl- | 0.2 | −3.16 | −1.17 | ¹¹C | ¹⁸F |
| | | | cyclohexane | | | | | |
| 18 | (bromomethoxyphenyl)acetyl | H | 1-amino4-fluoroethyl- | 1.24 | −2.13 | −1.17 | ⁷⁶Br, | ¹⁸F |
| | | | cyclohexane | | | | ¹¹C | |
| 19 | (iodomethoxyphenyl)acetyl | H | 1-amino4-fluoroethyl- | 1.46 | −1.91 | −1.17 | ¹²³I, ¹²⁴I, ¹³¹I, | ¹⁸F |
| | | | cyclohexane | | | | ¹¹C | |
| 20 | (methoxyphenyl)acetyl | t-Butyl | 1-amino4-fluoroethyl- | 5.13 | −0.48 | −1.17 | ¹¹C | ¹⁸F |
| | | | cyclohexane | | | | | |
| 21 | (bromomethoxyphenyl)acetyl | t-Butyl | 1-amino4-fluoroethyl- | 6.17 | 0.55 | −1.17 | ⁷⁶Br, | ¹⁸F |
| | | | cyclohexane | | | | ¹¹C | |
| 22 | (iodomethoxyphenyl)acetyl | t-Butyl | 1-amino4-fluoroethyl- | 6.39 | 0.77 | −1.17 | ¹²³I, ¹²⁴I, ¹³¹I, | ¹⁸F |
| | | | cyclohexane | | | | ¹¹C | |
| 23 | (methoxyphenyl)acetyl | H | aniline | 0.24 | −3.16 | 0.94 | ¹¹C | |
| 24 | (bromomethoxyphenyl)acetyl | H | aniline | 0.8 | −2.13 | 0.94 | ⁷⁶Br, | |
| | | | | | | | ¹¹C | |
| 25 | (iodomethoxyphenyl)acetyl | H | aniline | 1.02 | −1.91 | 0.94 | ¹²³I, ¹²⁴I, ¹³¹I, | |
| | | | | | | | ¹¹C | |
| 26 | (methoxyphenyl)acetyl | t-Butyl | aniline | 4.75 | −0.48 | 0.94 | ¹¹C | |
| 27 | (bromomethoxyphenyl)acetyl | t-Butyl | aniline | 5.78 | 0.55 | 0.94 | ⁷⁶Br, | |
| | | | | | | | ¹¹C | |
| 28 | (iodomethoxyphenyl)acetyl | t-Butyl | aniline | 6.01 | 0.77 | 0.94 | ¹²³I, ¹²⁴I, ¹³¹I, | |
| | | | | | | | ¹¹C | |
| 29 | (methoxyphenyl)acetyl | H | p-fluoroaniline | 0.13 | −3.16 | 1.15 | ¹¹C | ¹⁸F |
| 30 | (bromomethoxyphenyl)acetyl | H | p-fluoroaniline | 1.16 | −2.13 | 1.15 | ⁷⁶Br, | ¹⁸F |
| | | | | | | | ¹¹C | |
| 31 | (iodomethoxyphenyl)acetyl | H | p-fluoroaniline | 1.39 | −1.91 | 1.15 | ¹²³I, ¹²⁴I, ¹³¹I, | ¹⁸F |
| | | | | | | | ¹¹C | |
| 32 | (methoxyphenyl)acetyl | t-Butyl | p-fluoroaniline | 5.14 | −0.48 | 1.15 | ¹¹C | ¹⁸F |
| 33 | (bromomethoxyphenyl)acetyl | t-Butyl | p-fluoroaniline | 6.17 | 0.55 | 1.15 | ⁷⁶Br, | ¹⁸F |
| | | | | | | | ¹¹C | |
| 34 | (iodomethoxyphenyl)acetyl | t-Butyl | p-fluoroaniline | 6.4 | 0.77 | 1.15 | ¹²³I, ¹²⁴I, ¹³¹I, | ¹⁸F |
| | | | | | | | ¹¹C | |
| 35 | (methoxyphenyl)acetyl | H | p-fluoromethylaniline | −0.03 | −3.16 | 1.16 | ¹¹C | ¹⁸F |
| 36 | (bromomethoxyphenyl)acetyl | H | p-fluoromethylaniline | 1 | −2.13 | 1.16 | ⁷⁶Br, | ¹⁸F |
| | | | | | | | ¹¹C | |
| 37 | (iodomethoxyphenyl)acetyl | H | p-fluoromethylaniline | 1.22 | −1.91 | 1.16 | ¹²³I, ¹²⁴I, ¹³¹I, | ¹⁸F |
| | | | | | | | ¹¹C | |
| 38 | (methoxyphenyl)acetyl | t-Butyl | p-fluoromethylaniline | 4.98 | −0.48 | 1.16 | ¹¹C | ¹⁸F |
| 39 | (bromomethoxyphenyl)acetyl | t-Butyl | p-fluoromethylaniline | 6.01 | 0.55 | 1.16 | ⁷⁶Br, | ¹⁸F |
| | | | | | | | ¹¹C | |
| 40 | (iodomethoxyphenyl)acetyl | t-Butyl | p-fluoromethylaniline | 6.23 | 0.77 | 1.16 | ¹²³I, ¹²⁴I, ¹³¹I, | ¹⁸F |
| | | | | | | | ¹¹C | |
| 41 | (methoxyphenyl)acetyl | H | p-fluorobenzylamine | −0.43 | −3.16 | −0.75 | ¹¹C | ¹⁸F |
| 42 | (bromomethoxyphenyl)acetyl | H | p-fluorobenzylamine | 0.6 | −2.13 | −0.75 | ⁷⁶Br, | ¹⁸F |
| | | | | | | | ¹¹C | |
| 43 | (iodomethoxyphenyl)acetyl | H | p-fluorobenzylamine | 0.83 | −1.91 | −0.75 | ¹²³I, ¹²⁴I, ¹³¹I, | ¹⁸F |
| | | | | | | | ¹¹C | |
| 44 | (methoxyphenyl)acetyl | t-Butyl | p-fluorobenzylamine | 4.51 | −0.48 | −0.75 | ¹¹C | ¹⁸F |
| 45 | (bromomethoxyphenyl)acetyl | t-Butyl | p-fluorobenzylamine | 5.54 | 0.55 | −0.75 | ⁷⁶Br, | ¹⁸F |
| | | | | | | | ¹¹C | |
| 46 | (iodomethoxyphenyl)acetyl | t-Butyl | p-fluorobenzylamine | 5.77 | 0.77 | −0.75 | ¹²³I, ¹²⁴I, ¹³¹I, | ¹⁸F |
| | | | | | | | ¹¹C | |
| 47 | (methoxyphenyl)acetyl | H | p- | −0.25 | −3.16 | −0.52 | ¹¹C | ¹⁸F |
| | | | fluoromethylbenzylamine | | | | | |
| 48 | (bromomethoxyphenyl)acetyl | H | p- | 0.78 | −2.13 | −0.52 | ⁷⁶Br, | ¹⁸F |
| | | | fluoromethylbenzylamine | | | | ¹¹C | |
| 49 | (iodomethoxyphenyl)acetyl | H | p- | 1.01 | −1.91 | −0.52 | ¹²³I, ¹²⁴I, ¹³¹I, | ¹⁸F |
| | | | fluoromethylbenzylamine | | | | ¹¹C | |
| 50 | (methoxyphenyl)acetyl | t-Butyl | p- | 4.68 | −0.48 | −0.52 | ¹¹C | ¹⁸F |
| | | | fluoromethylbenzylamine | | | | | |
| 51 | (bromomethoxyphenyl)acetyl | t-Butyl | p- | 5.71 | 0.55 | −0.52 | ⁷⁶Br, | ¹⁸F |
| | | | fluoromethylbenzylamine | | | | ¹¹C | |
| 52 | (iodomethoxyphenyl)acetyl | t-Butyl | p- | 5.94 | 0.77 | −0.52 | ¹²³I, ¹²⁴I, ¹³¹I, | ¹⁸F |
| | | | fluoromethylbenzylamine | | | | ¹¹C | |
| 53 | (methoxyphenyl)acetyl | H | 4-aminopiperidine | −1.16 | −3.16 | −3.87 | ¹¹C | |
| 54 | (bromomethoxyphenyl)acetyl | H | 4-aminopiperidine | −0.13 | −2.13 | −3.87 | ⁷⁶Br, | |
| | | | | | | | ¹¹C | |
| 55 | (iodomethoxyphenyl)acetyl | H | 4-aminopiperidine | 0.096 | −1.91 | −3.87 | ¹²³I, ¹²⁴I, ¹³¹I, | |
| | | | | | | | ¹¹C | |
| 56 | (methoxyphenyl)acetyl | t-Butyl | 4-aminopiperidine | 0.43 | −0.48 | −3.87 | ¹¹C | |
| 57 | (bromomethoxyphenyl)acetyl | t-Butyl | 4-aminopiperidine | 1.46 | 0.55 | −3.87 | ⁷⁶Br, | |
| | | | | | | | ¹¹C | |
| 58 | (iodomethoxyphenyl)acetyl | t-Butyl | 4-aminopiperidine | 1.69 | 0.77 | −3.87 | ¹²³I, ¹²⁴I, ¹³¹I, | |
| | | | | | | | ¹¹C | |
| 59 | (methoxyphenyl)acetyl | H | 4-amino-1- | −0.6 | −3.16 | −2.53 | ¹¹C | ¹⁸F |
| | | | fluoroethylpiperidine | | | | | |
| 60 | (bromomethoxyphenyl)acetyl | H | 4-amino-1- | 0.44 | −2.13 | −2.53 | ⁷⁶Br, | ¹⁸F |
| | | | fluoroethylpiperidine | | | | ¹¹C | |
| 61 | (iodomethoxyphenyl)acetyl | H | 4-amino-1- | 0.66 | −1.91 | −2.53 | ¹²³I, ¹²⁴I, ¹³¹I, | ¹⁸F |
| | | | fluoroethylpiperidine | | | | ¹¹C | |
| 62 | (methoxyphenyl)acetyl | t-Butyl | 4-amino-1- | 3.43 | −0.48 | −2.53 | ¹¹C | ¹⁸F |
| | | | fluoroethylpiperidine | | | | | |
| 63 | (bromomethoxyphenyl)acetyl | t-Butyl | 4-amino-1- | 4.46 | 0.55 | −2.53 | ⁷⁶Br, | ¹⁸F |
| | | | fluoroethylpiperidine | | | | ¹¹C | |
| 64 | (iodomethoxyphenyl)acetyl | t-Butyl | 4-amino-1- | 4.66 | 0.77 | −2.53 | ¹²³I, ¹²⁴I, ¹³¹I, | ¹⁸F |
| | | | fluoroethylpiperidine | | | | ¹¹C | |
| 65 | (methoxyphenyl)acetyl | H | 4-amino-1- | −0.14 | −3.16 | −2.28 | ¹¹C | ¹⁸F |
| | | | fluoropropylpiperidine | | | | | |
| 66 | (bromomethoxyphenyl)acetyl | H | 4-amino-1- | 0.89 | −2.13 | −2.28 | ⁷⁶Br, | ¹⁸F |
| | | | fluoropropylpiperidine | | | | ¹¹C | |
| 67 | (iodomethoxyphenyl)acetyl | H | 4-amino-1- | 1.11 | −1.91 | −2.28 | ¹²³I, ¹²⁴I, ¹³¹I, | ¹⁸F |
| | | | fluoropropylpiperidine | | | | ¹¹C | |
| 68 | (methoxyphenyl)acetyl | t-Butyl | 4-amino-1- | 3.43 | −0.48 | −2.28 | ¹¹C | ¹⁸F |
| | | | fluoropropylpiperidine | | | | | |
| 69 | (bromomethoxyphenyl)acetyl | t-Butyl | 4-amino-1- | 4.46 | 0.55 | −2.28 | ⁷⁶Br, | ¹⁸F |
| | | | fluoropropylpiperidine | | | | ¹¹C | |
| 70 | (iodomethoxyphenyl)acetyl | t-Butyl | 4-amino-1- | 4.66 | 0.77 | −2.28 | ¹²³I, ¹²⁴I, ¹³¹I, | ¹⁸F |
| | | | fluoropropylpiperidine | | | | ¹¹C | |
| 71 | (methoxyphenyl)acetyl | H | p-nitroaniline | 0.39 | −3.16 | 1.39 | ¹¹C | |
| 72 | (bromomethoxyphenyl)acetyl | H | p-nitroaniline | 1.43 | −2.13 | 1.39 | ⁷⁶Br, | |
| | | | | | | | ¹¹C | |
| 73 | (iodomethoxyphenyl)acetyl | H | p-nitroaniline | 1.65 | −1.91 | 1.39 | ¹²³I, ¹²⁴I, ¹³¹I, | |
| | | | | | | | ¹¹C | |
| 74 | (methoxyphenyl)acetyl | t-Butyl | p-nitroaniline | 5.33 | −0.48 | 1.39 | ¹¹C | |
| 75 | (bromomethoxyphenyl)acetyl | t-Butyl | p-nitroaniline | 6.36 | 0.55 | 1.39 | ⁷⁶Br, | |
| | | | | | | | ¹¹C | |
| 76 | (iodomethoxyphenyl)acetyl | t-Butyl | p-nitroaniline | 6.57 | 0.77 | 1.39 | ¹²³I, ¹²⁴I, ¹³¹I, | |
| | | | | | | | ¹¹C | |
| 77 | (fluoromethoxyphenyl)acetyl | H | p-nitroaniline | 0.52 | −3.03 | 1.39 | ¹¹C, ¹⁸F | |
| 78 | (fluoromethylmethoxyphenyl)acetyl | H | p-nitroaniline | 0.62 | −2.94 | 1.39 | ¹¹C, ¹⁸F | |
| 79 | (fluoroethylmethoxyphenyl)acetyl | H | p-nitroaniline | 0.66 | −2.9 | 1.39 | ¹¹C, ¹⁸F | |
| 80 | (methylmethoxyphenyl)acetyl | H | p-nitroaniline | 0.85 | −2.7 | 1.39 | ¹¹C | |
| 81 | (fluoromethoxyphenyl)acetyl | t-Butyl | p-nitroaniline | 5.46 | −3.03 | 1.39 | ¹¹C, | |
| | | | | | | | ¹⁸F | |
| 82 | (fluoromethylmethoxyphenyl)acetyl | t-Butyl | p-nitroaniline | 5.55 | −2.94 | 1.39 | ¹¹C, | |
| | | | | | | | ¹⁸F | |
| 83 | (fluoroethylmethoxyphenyl)acetyl | t-Butyl | p-nitroaniline | 5.59 | −2.9 | 1.39 | ¹¹C, | |
| | | | | | | | ¹⁸F | |
| 84 | (methylmethoxyphenyl)acetyl | t-Butyl | p-nitroaniline | 5.79 | −2.7 | 1.39 | ¹¹C | |

Generally, the imaging agents of the present disclosure can be synthesized using solid phase Fmoc chemistry. Any other suitable synthesis route, however, may be used.

The present disclosure also relates to processes for imaging the imaging agents represented by Formula I, among other thing, to measure the activity of intracellular caspases or other enzymes involved in apoptosis in living or dead whole cells or tissues. The method for imaging caspase activity in cells or tissues of a mammal comprises contacting the cells or tissues with an imaging agent of Formula I and detecting caspase activity using molecular imaging. The term, “contacting the cells or tissues” may refer to causing the imaging agents of the present disclosure to come into contact with a cell or tissue such that the imaging agent may diffuse into the cell or tissue, and in animals also may refer to administering the imaging agents of the present disclosure to an animal by, for example, injection. To assess caspase activity, the labeled cleaved product of the imaging agent may be detected by imaging the cells or tissues using imaging techniques known in the art (e.g., BLI imaging, PET imaging, fluorescent imaging, and the like). The choice of imaging technique to detect caspase cell activity depends upon the radiolabeling or fluorescent capabilities of the imaging agent. The imaging agent can also be used to measure baseline caspase activity in cells that are not undergoing apoptosts.

For example, the imaging agent may be used in applications for determining whether a test substance has an effect on an enzyme involved in the apoptosis cascade in a test cell. This method may involve contacting a test cell with the test substance and the imaging agent of the present disclosure under conditions wherein the test substance either interacts with the external membrane of the cell or is taken into the cell and the imaging agent is taken into the cell, imaging the test cell to detect the accumulation of the labeled cleavage product of the imaging agent in the test cell compared to a control test cell which has only been contacted with the imaging agent. Such methods may indicate whether the test substance had an effect on the enzyme of interest. A test cell could be a cancer cell or cell line derived from a human in need of treatment with a chemotherapeutic drug and the test substance could be a chemotherapeutic agent or drug or a mixture of chemotherapeutic agents or drugs. In the practice of this aspect of the present disclosure, the test cells may be contacted with the test substance prior to, after, or substantially simultaneously with the imaging agent. The method may be used to detect whether the test substance stimulates or inhibits the activity of the enzyme.

The compounds described herein arc intended to include salts, cnantiomcrs, esters, and hydrates, in pure form and as a mixture thereof. Also, when a nitrogen atom appears, it is understood sufficient hydrogen atoms are present to satisfy the valency of the nitrogen atom.

While chiral structures may be shown above, by substituting into the synthesis schemes an enantiomer other than the one shown, or by substituting into the schemes a mixture of enantiomers, a different isomer or racemic mixture can be achieved. Thus, all such isomers and mixtures are included in the present disclosure. The compounds described may contain asymmetric centers and may thus give rise to diastereomers and optical isomers, the present disclosure is meant to comprehend such possible diastereomers as well as their racemic and resolve, enantiomerically pure forms and pharmaceutically acceptable salts thereof. Some of the compounds described herein may contain olefinic double bonds, and unless specified otherwise, arc meant to include both E and Z geometric isomers.

Pharmaceutical compositions may be utilized to administer the compounds of the present disclosure. Such pharmaceutical compositions comprise a compound of Formula I in combination with a pharmaceutically acceptable carrier, and optionally other therapeutic ingredients. The term “salts” refers to salts prepared from pharmaceutically acceptable bases including inorganic bases and organic bases. Representative salts derived from inorganic bases include aluminum, ammonium, calcium, copper, ferric, ferrous, lithium, magnesium, manganic salts, manganous, ammonium, potassium, sodium, zinc, and the like. Particularly preferred are the calcium, magncsium, potassium, and sodium salts. Representative salts derived from pharmaceutically acceptable organic bases include salts of primary, secondary and tertiary amines, substituted amines including naturally occurring substituted amines, cyclic amines, and basic ion exchange resins, such as arginine, betaine, caffeine, choline, NN′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabarmine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present disclosure is basic, salts may be prepared from pharmaceutically acceptable non-toxic acids, including inorganic and organic acids. Examples of such acids include acetic, benzenesulfonic, benzoic, camphorsulfonic, citric, ethanesulfonic, fumaric, gluconic, glutamic, hydrobromic, hydrochloric, iscthionic, lactic, malcic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric and tartaric acids.

The magnitude of dose of a compound of Formula I will, of course, vary depending on the particular application, desired result, and the particular compound of Formula I. Representative amounts of the compounds for use in the present disclosure are in the examples that follow.

Any suitable route of administration may be employed for providing a dosage of a compound of the present disclosure. For example, oral, parenteral and topical may be employed. Dosage forms include tablets, troches, dispersions, suspensions, solutions, capsules, creams, ointments, aerosols, and the like.

To facilitate a better understanding of the present disclosure, the following examples of specific embodiments are given. In no way should the following examples be read to limit or define the entire scope of the invention.

### EXAMPLES

Researchers at Merck Research Laboratories (Montreal, Canada) reported in Method, N., et al., J. Biol. Chem. 279:27905-14 (2004). a novel radiolabeled, irreversible caspase inhibitor [¹²⁵I]M808 which acts as a caspase-3 active site probe. [¹²⁵I]M808 bound to active caspases irreversibly and with high sensitivity in apoptotic cell extracts, in tissue extracts from several commonly used animal models of cellular injury, and in living cells. Moreover, [¹²⁵I]M808 detected active caspases in septic mice when injected intravenously. Using this caspase probe, an active site occupancy assay was developed and used to measure the fractional inhibition required to block apoptosis-induced DNA fragmentation. In thymocytes, occupancy of up to 40% of caspase active sites had no effect on DNA fragmentation, whereas inhibition of half of the DNA cleaving activity required between 65 and 75% of active site occupancy. These results suggested that a high and persistent fractional inhibition will be required for successful caspase inhibition-based therapies. However, the uneven distribution of [¹²⁵I]M808 precluded determination of caspase active site occupancy in vivo, and no non-invasive imaging has been attempted with [¹²⁵I]M808. The inhibitory profile of M808 against purified recombinant caspases indicated a slight preference (2-fold) for caspasc-3 over caspasc-8, and a 10-fold preference over caspasc-7. Another compound previously reported by researchers at Merck is M826.

To develop a novel caspase imaging agent based on one of the Merck caspase-3 inhibitor M808, M808 was converted into a substrate for caspase-3, and the process of developing several radiolabeled derivatives is underway. To establish a proof-of-principle, AMC-M808 was initially synthesized, in which AMC fluorescence is quenched by M808. Phe(2-OMe)CH₂CO-Val was synthesized using Fmoc solid-phase chemistry and cleaved with TFA/H20/TES (95/1/4, v/v/v) from resin. After purification from HPLC Phe(2-OMe)CH₂CO-Val was treated with Asp(Bzl)-AMC using PyBOP (1.5 eq), HOBt (1.5 eq), and DIPEA (3 eq) as coupling agents in DMF. The product was purified by HPLC.

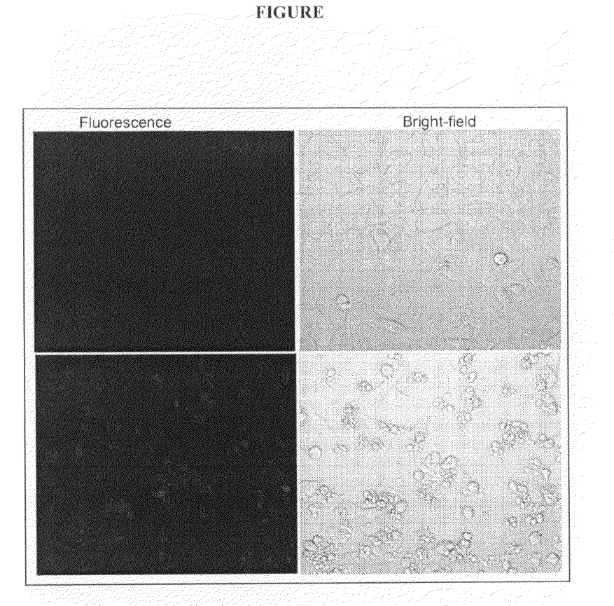

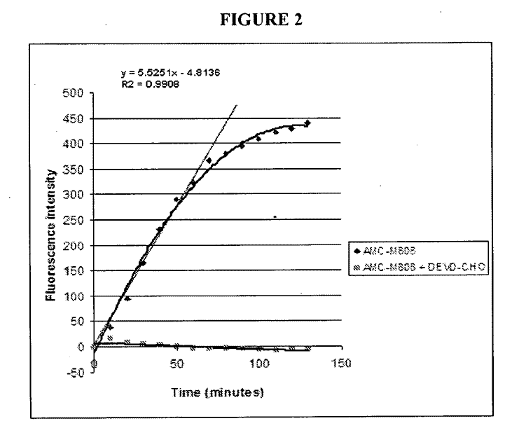

In DLD1 colon carcinoma cells in vitro, it was demonstrated that the AMC-M808 is cell permeable and is cleaved by caspase-3 after induction of apoptosis by treatment of these cells with TNF-related apoptosis-inducing ligand (TRAIL) (FIG. 1). DLD1 cells were seeded in an 8-chamber glass slide at a density of 2×10⁴ cells/cm² in DMEM containing 10% FBS and allowed to attach overnight. Fluorescent images of the plated cells were acquired using an Olympus FV1000 confocal microscope equipped with an onstage incubation chamber. TRAIL was added to the cells in a 0.5 mL volume of fresh medium at a concentration of 50 ng/mL. Time-lapse microscopy was then immediately initiated with confocal images acquired at a rate of one image every 10 minutes. The cells were maintained at 37° C. in 5% CO₂ for the duration of the experiment. An excitation wavelength of 405 nm and an emission wavelength range of 425 to 475 nm were used to detect the cleaved product of AMC-M808. Fluorescence intensity over time was measured in a representative region-of-interest in each 10-minute image acquired to determine the rate of caspase activity. In a separate experiment, cells were preincubated with a cell-permeable caspase-3 inhibitor, DEVD-CHO (Calbiochem), at a 50 μM concentration for 1 hour before adding TRAIL to assess the specificity of AMC-M808. The rate of cleavage following TRAIL addition was fast and fluorescence intensity in the apoptotic cell population increased over time at a rate of 5.5 min⁻¹ indicating accumulation and retention of the cleaved substrate (FIG. 2). In the presence of DEVD-CHO, no net increase was observed in the fluorescence intensity, demonstrating the specificity of AMC-M808 for caspase-3.

The next series of cell permeable caspase-3 substrate-like molecular imaging agents were based on M808 pharmacophore, which was derivatized with various, more hydrophilic groups linked to the terminal Asp with the basic structure: R₁-Val-Asp-(O-R₂)-R₃ (Table 2). Compounds 1, 3, 8, 53, and 56 have been synthesized.

Methoxylphenylacetyl seems to have reasonable alignment with Asp-Glu- of Ac-DEVD-R. Compound 59 of Table 2 contains a piperidine leaving group, which is the most lipophilic when cleaved (logD −3.16). However, the intact compound itself may not be lipophilic enough to cross cell membranes by non-facilitated diffusion (logD −0.06). In contrast, compound 60 of Table 2 has a higher lipophilicity (logD 0.44), and a hydrolyzed radiolabeled leaving group with a logD less than 0 (logD −2.53).

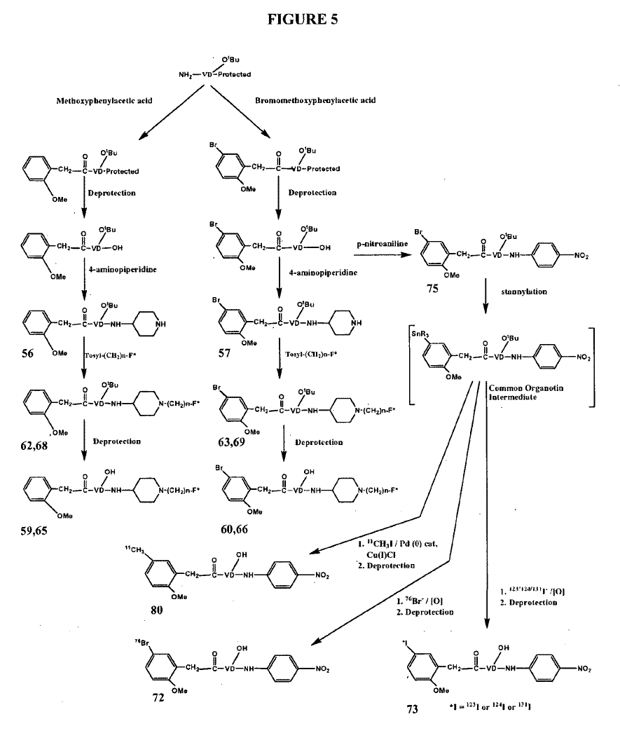

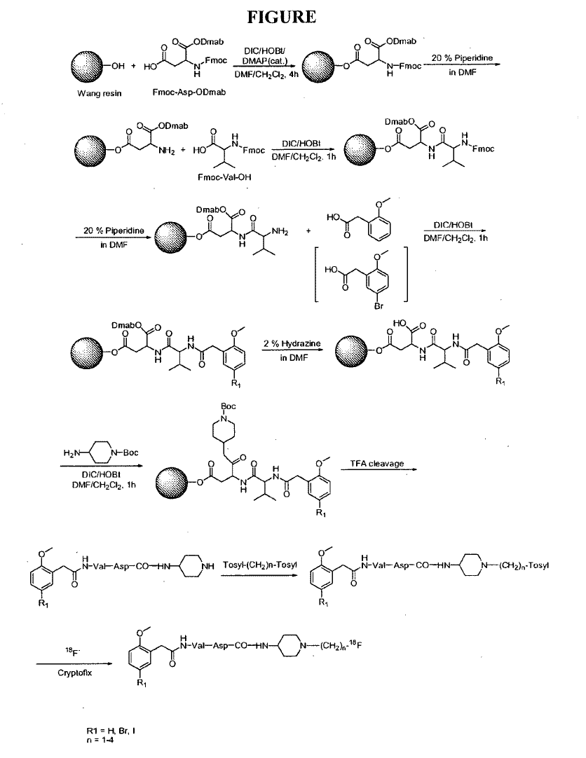

The synthesis of Z-VD-piperidine was synthesized via standard solid phase Fmoc chemistry (FIGS. 5 and 6). The side-chain carboxyl group of Fmoc-Asp-ODmab was first attached to the Wang resin using diisopropylcarbodiimide (DIC) and 1-hydroxylbenzotriazole (HOBt) as coupling agents and 0.1 equivalent of 4-(dimethylamino)-pyridine (DMAP) as a base catalyst. Fmoc-Val-OH and 2-methoxyphenyl acetic acid were the sequentially coupled to the resin via standard solid phase Fmoc chemistry. The extent of each acylation was monitored via standard ninhydrin test. The Fmoc groups were deprotected with 20% piperidine solution in DMF (v/v) after each coupling step. The Dmab group was selectively removed by treating with 2% hydrazine in DMF (v/v) for 4 min. BOC-piperidine was conjugated to C-terminus carboxyl group on solid resin using DIC/HOBt as the coupling reagent. Cleavage of peptides from the resin was completed with TFA/CH₂Cl₂ (l/l, v/v) for 3 h. After the cleavage, TFA in the residue was removed with a N₂ strcam and cold ether was added to the residue to precipitate the cnide product. The crude product was collected via centrifuging followed by lyophilization. The product was characterized by LC-MS.

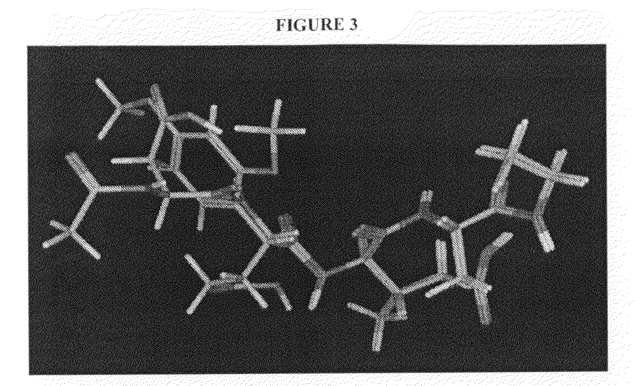

The alignment of two molecules, acetyl-DEVD-fluoroethylamide to dimethoxyphenylacetate-VD-fluoroethylamide, is shown in FIG. 3. Fluorine is green, oxygen is red and nitrogen is blue. Since VD-fluoroethylamides are identical (right side), the alignment of Acetyl-DE to dimethoxyphenylacetate is very good (left side).

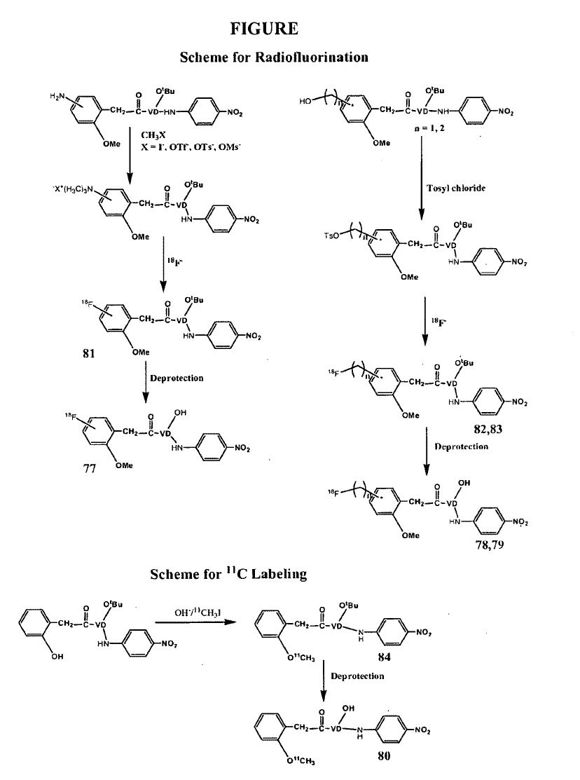

Example radiolabeling schemes for ¹⁸F radiofluorination and ¹¹C are shown in FIG. 7.

The uptake and retention of ¹⁸F-radiolabeled leaving group in vitro can be tested in DLD1 tumor cells. Similarly, the uptake and retention of AMC-M80 can be tested by in vivo PET imaging studies in mice bearing DLD1 tumor xenografts.

Notwithstanding that the numerical ranges and parameters setting forth the broad scope of the invention arc approximations, the numerical values set forth in the specific examples arc reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

### REFERENCES

Krammer, P. H. 2000. CD95's deadly mission in the immune system. Nature. 407:789-795.

Danial, N. N., and Korsmeyer, S. J. 2004. Cell death: critical control points. Cell. 116:205-219.

Ashkenazi, A., and Dixit, V. M. 1998. Death receptors: signaling and modulation. Science. 281:1305-1308.

Krammer, P. H. 1998. The CD95(APO-1/Fas)/CD95L system [review]. Toxicol. Lett. 102-103:131-137.

Thomberry, N. A., and Lazebnik, Y. 1998. Caspases: enemies within. Science. 281:1312-1316.

Cohen, G. M. 1997. Caspases: the executioners of apoptosis. Biochem. J. 326:1-16.

Nicholson, D. W. 1999. Caspase structure, proteolytic substrates, and function during apoptotic cell death. Cell Death Differ. 6:1028-1042.

Alnemri, E. S. et al. 1996. Human ICE/CED-3 protease nomenclature [letter]. Cell. 87:171.

Thornberry, N. A. et al. 1997. A combinatorial approach defines specificities of members of the caspase family and granzyme B. Functional relationships established for key mediators of apoptosis. J. Biol. Chem. 272:17907-17911.

Boatright, K. M. et al. 2003. A unified model for apical caspase activation. Mol. Cell. 11:529-541.

Shi, Y. 2002. Mechanisms of caspase activation and inhibition during apoptosis. Mol. Cell. 9:459-470.

Graybill, T. L., Dolle, R. E., Hclaszek, C. T., Miller, R. E., and Ator, M. A. 1994. Preparation and evaluation of peptidic aspartyl hemiacetals as reversible inhibitors of interleukin-1 beta converting enzyme (ICE). Int. J. Pept. Protein Res. 44:173-182.

Estrov, Z. et al. 1995. Effect of interleukin-I beta converting enzyme inhibitor on acute myelogenous leukemia progenitor proliferation. Blood. 86:4594-4602.

Thomberry, N. A. et al. 1992. A novel heterodimeric cysteine protease is required for interleukin-1 beta processing in monocytes. Nature. 356:768-774.

Han, B. H., Xu, D., Choi, J., Han, Y., Xanthoudakis, S., Roy, S., Tam, J., Vaillancourt, J., Colucci, J., Siman, R., Giroux, A., Robertson, G., Zamboni, R., Nicholson, D. W., and Holtzman, D. M. (2002) J. Biol. Chem. 277, 30128-30136.

Haberkorn U, Kinscherf R, Krammer PH, Mier W, Eisenhut M. Investigation of a potential scintigraphic marker of apoptosis: radioiodinated Z-Val-Ala-DL-Asp(O-methyl)-fluoromethyl ketone. Nucl Med Biol. 2001 October; 28(7):793-8.

Bauer C, Bauder-Wuest U, Mier W, Haberkorn U, Eisenhut M. ¹³¹I-labeled peptides as caspase substrates for apoptosis imaging. J Nucl Med. 2005 June; 46(6): 1066-74.

What is claimed is:

1. A composition comprising the following formula: 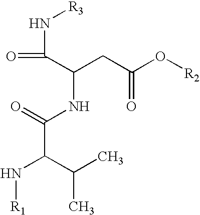
or salt, ester, or hydrate thereof wherein:
R₁ is selected from the group consisting of Asp-Glu peptide, tert-butyloxycarbonyl, methoxyphenylacetyl, bromomethoxyphenylacetyl, fluoromethoxyphenylacetyl, (methylmethoxyphenyl)acetyl, and iodomethoxyphenylacetyl;
R₂ is selected from the group consisting of valine (with or without protecting groups), aspartate (with or without protecting groups), t-Butyl, hydrogen, aniline, aromatic molecules, tert-butyloxycarbonyl, and fluorenyl-methoxy-carbonyl; and
R₃ is selected from the group consisting of p-nitroaniline, 7-amino-4-methylcoumarin, fluoroethylamine, 1-amino-4-fluoromethyl-cyclohexane, 1-amino-4-fluoroethyl-cyclohexane, aniline, p-fluoroaniline, p-fluoromethylaniline, p-fluorobenzylamine, p-fluorotnethylbenzylamine, 4-aminopiperidine, 4-amino-1-fluoroethylpiperidine, and 4-amino-1-fluoropropylpiperidine.

2. The composition of claim 1 wherein R₁ is radiolabeled or fluorescently labeled or both.

3. The composition of claim 1 wherein R₁ is radiolabeled or fluorescently labeled or both.

4. The composition of claim 1 wherein R₁ is radiolabeled with one or more of ⁷⁶Br, ¹¹C, ¹⁸F, ¹²³I, ¹²⁴I, and ¹³¹I.

5. The composition of claim 1 wherein R₃ is radiolabeled with one or more of ⁷⁶Br, ¹¹C, ¹⁸F, ¹²³I, ¹²⁴I, and ¹³¹I.

6. The composition of claim 1 wherein R₂ enhances the lipophlicity of the imaging agent.

7. The composition of claim 1 having a logD greater than zero.

8. A composition comprising an a compound according to claim 1 cleaved by a caspsase.

9. A method of detecting active caspase-3 activity in cells or tissues of a mammal comprising contacting the cells or tissues with an imaging agent of claim 1 wherein one or more of R₁ and R₃ are independently radiolabeled or fluorescently labeled or both, and detecting active caspase-3.

10. The method of claim 9 wherein active caspase-3 cleaves the imaging agent forming a labeled product.

11. The method of claim 9 wherein active caspase-3 cleaves the imaging agent forming a labeled product, wherein the labeled product is radiolabeled or capable of fluorescence, or both.

12. A method of molecular imaging of apoptosis comprising:
contacting cells or tissues with an imaging agent of claim 1 wherein one or more of R₁ and R₃ are independently radiolabeled or fluorescently labeled or both;
allowing the imaging agent to be cleaved by active caspase enzyme to form a labeled product;
allowing the labeled product to accumulate in the cells or tissues; and
imaging the cells or tissues to detect the labeled product or products.

13. The method of claim 12 wherein the labeled product is radiolabeled or fluorescent or both.

14. The method of claim 12 wherein the caspase enzyme is caspase-3.

15. The method of claim 12 wherein imaging of the cells or tissues is performed using one or more of BLI imaging, PET imaging, and fluorescent imaging.

16. The method of claim 12 wherein the imaging agent is a substrate of caspase-3.

17. The method of claim 12 wherein the labeled product has a logD of less than zero.

18. The method of claim 12 wherein the labeled product has a logD of greater than zero.

19. A method of assessing the effect of a test substance on an enzyme effecting apoptosis comprising:
contacting the test cell with the test substance and a imaging agent according to claim 1 under conditions wherein the test substance either interacts with the external membrane of the cell or is taken into the cell; and
imaging the test cell to detect the accumulation of labeled products of the imaging agent in the test cell compared to a control test cell which has only been contacted with the imaging agent.

20. The method of claim 19 wherein the test cell is a cancer cell.

21. The method of claim 19 wherein the test substance is a chemotherapeutic agent, chemotherapeutic drug, or combinations thereof.
